(19) United States
(12) Patent Application Publication
(10) Pub. No.: US 2014/0105871 A1
(43) Pub. Date: Apr. 17, 2014

(54) Use Of Mesenchymal Stem Cells For The Improvement Of Affective And Cognitive Function

(71) Applicant: Ariel University Research and Development Company, Ltd., Ariel (IL)

(72) Inventors: Gadi Turgeman, D.N. East Binyamin (IL); Matanel Tfilin, Shaarey-Tikva (IL); Nikoli Gobshtise, Ariel (IL)

(73) Assignee: Ariel University Research and Development Company, Ltd., Ariel (IL)

(21) Appl. No.: 14/084,306

(22) Filed: Nov. 19, 2013

Related U.S. Application Data
(63) Continuation of application No. PCT/IL2012/000195, filed on May 17, 2012.
(60) Provisional application No. 61/488,035, filed on May 19, 2011.

(51) Int. Cl. A61K 35/28; C12N 5/0775

(52) U.S. Cl. CPC A61K 35/28; C12N 5/0675
USPC 424/93.7; 435/375

(57) ABSTRACT

Disclosed is the use of mesenchymal stem cells (MSCs) and in particular MSCs pretreated with pituitary adenylate cyclase-activating polypeptide (PACAP) or analogs and fragments of PACAP, for treatment of neurodegenerative and psychiatric diseases, and for the improvement of affective and cognitive function in a normal individual or in an individual suffering from a neurodegenerative or neuropsychiatric disease.

### FIELD OF THE INVENTION

The invention, in some embodiments, relates to the field of stem cell therapy and more particularly, but not exclusively, to the use of mesenchymal stem cells for the treatment of neurodegenerative and psychiatric diseases, and for improvement of affective and cognitive behavior.

### BACKGROUND OF THE INVENTION

Neurodegenerative diseases are generally considered to be incurable diseases in which the nervous system is irreversibly impaired by the death and loss of functional neurons. Psychiatric diseases such as major depression and schizophrenia are also considered to be incurable, and require chronic medical management. Such diseases accompanied by the loss of cognitive and affective behavioral functions result in the patient losing independence and social acceptability and prolonged hospitalization is often required. Thus, these diseases impose a major burden on healthcare services. Both cognitive and affective behaviors impaired in these diseases are associated with a significant decrease in neurogenesis in the hippocampus, and can therefore be a target for stem cell therapy.

Schizophrenia is a disorder characterized by disturbances in perception, thought, volition, socialization, psychomotor behavior and the sense of self. Among clinicians and investigators there is little doubt that, in the majority of patients, schizophrenia runs a progressive course. Patients suffering from schizophrenia start from a point of relative normalcy or subtle impairment. Following the formal onset most patients experience, to some degree, what has been called clinical deterioration. This deterioration is manifest by the development of and increasing severity and persistence of positive symptoms such as delusional behavior and/or psychotic episodes, negative symptoms such as diminished social and functional capacity, and cognitive impairment.

Mesenchymal Stem Cells (MSCs) are easily isolated, pluripotent stem cells, which are able to differentiate into a variety of lineages, including neural lineages. Moreover, MSCs can express and secrete neurotrophic factors that promote the survival and differentiation of neural cells and can interact with the immune system playing a role in some of the above-mentioned diseases. Thus, MSCs are good candidates for treatment of neurodegenerative disorders.

The present inventor and co-workers have previously discovered that bone-marrow derived MSCs can enhance neurogenesis in the dentate gyrus (hippocampus) and reverse depressive-type behavior, particularly affective behavior in a rat model for depression, i.e. forced swim test and dominant submissive relations (Tfilin M et al. Molecular Psychiatry, Volume: 15 Issue: 12 Pages: 1164-1175, 2010, which is incorporated by reference as if fully set forth herein).

US Patent Application No. 2012/0009673 discloses an isolated human cell comprising at least one mesenchymal stem cell phenotype and secreting brain-derived neurotrophic factor (BDNF), wherein a basal secretion of the BDNF is at least five times greater than a basal secretion of the BDNF in a mesenchymal stem cell. The application further discloses methods of generating said human cells and methods of treating a neurodegenerative disease or disorder.

US Patent Application No. 2010/0015105 provides a method of treating schizophrenia in a subject in need thereof, comprising administering to the subject a therapeutically effective amount of cells expressing at least one exogenous polypeptide forming a connexin channel and/or a hyperpolarizing ion channel.

There remains an unmet need for improved compositions and methods for treating neurodegenerative or psychiatric disorders, and for improvement of affective and cognitive behavior.

### SUMMARY OF THE INVENTION

The present invention provides cell based compositions comprising isolated mesenchymal stem cells (MSCs), methods of generating same and use thereof for treating neurodegenerative diseases or psychiatric disorders and for improving cognitive and affective function of a subject in need thereof. The present invention further provides methods for enhancing the neurogenic activity of said MSCs.

It is now disclosed for the first time that pretreatment of MSCs in culture with a fragment of pituitary adenylate cyclase-activating polypeptide (PACAP), dehydroepiandrosterone (DHEA) or lithium chloride (LiCl) can enhance their neurogenic activity. Transplantation of MSCs pre-treated with PACAP, via direct injection into the lateral ventricle improved the cognitive functions of normal mice. Surprisingly, a significant effect was also achieved when said MSCs were administered intravenously.

It is further disclosed that MSCs, injected directly into the lateral ventricle exhibit long term (at least three months) improvement in social preference and pre-pulse inhibition in murine model of schizophrenia. Further, MSCs administered intravenously, following incubation with PACAP, exhibited long term effect in pre-pulse inhibition in murine model of schizophrenia as well as short term effect on social preference. Notably, the results obtained with the MSCs surpass the effect of a 12 days course treatment with clozapine, a second generation antipsychotic.

Thus, the present invention provides, in some embodiments, cell based compositions comprising isolated MSCs having enhanced neurogenic activity. The MSCs neurogenic activity is enhanced, according to some embodiments, by incubating said MSCs with at least one agent selected from PACAP an analog, homolog or fragment thereof, DHEA or LiCl. In particular embodiments, the at least one agent is PACAP. In another embodiment, the PACAP comprises an amino acid sequence selected from the group consisting of: SEQ ID NO: 1, 2, 3, 4, 5, 6, 7, 8, 10 and 11. In another embodiment, the PACAP has an amino acid sequence selected from the group consisting of: SEQ ID NO: 1, 2, 3, 4, 5, 6, 7, 8, 10 and 11. In yet another embodiment, the PACAP is selected from the group consisting of: SEQ ID NO: 1, 2, 3 and 4. In another embodiment, the PACAP homolog is selected from the group consisting of SEQ ID NO: 5, 6, 7, 8, 10 and 11. Each possibility is a separate embodiment of the present invention.

In yet another particular embodiment, the at least one agent is a PACAP27. As used herein, PACAP27 refers to a fragment of 27 amino acid residues corresponding to amino acids 132-158 of human PACAP (Accession No: NP_{—}001093203.1), having the amino acid sequence as set forth in SEQ ID NO: 1 (HSDGIFTDSYSRYRKQMAVKKYLAAVL).

According to one aspect, there is provided cell based compositions comprising isolated mesenchymal stem cells (MSCs), for use in improving cognitive functions in a subject. According to another embodiment, there is provided a pharmaceutical composition comprising isolated MSCs for use in improving cognitive functions in a subject. According to another aspect, there is provides use of cell based compositions comprising isolated MSCs for preparation of a medicament for improving cognitive functions in a subject. According to some embodiments, the MSCs have been cultured with at least one agent selected from the group consisting of PACAP, DHEA and LiCl.

In another embodiment, the pharmaceutical composition are formulated (e.g., adapted) for intracerebroventricular (ICV) administration. In another embodiment, the pharmaceutical compositions are formulated for intravenous (IV) administration.

According to one aspect the present invention provides a method of improving cognitive function of a subject, the method comprising the step of administering to said subject a pharmaceutically effective amount of isolated MSCs, thereby improving the cognitive function of said subject. According to some embodiments, the method comprises administering to said subject a pharmaceutical composition comprising a pharmaceutically effective amount of the isolated MSCs.

According to additional embodiments, the method comprises pretreatment of the MSCs with at least one agent selected from the group consisting of PACAP, DHEA and LiCl.

As used herein “pretreatment of the MSCs” refers to the culturing (e.g., exposing, treating or contacting) of the isolated MSCs with the at least one agent, such as PACAP, prior to administration of the MSCs to a subject. According to some embodiments, the methods of the present invention further comprise, prior to administering the composition to the subject, contacting said MSCs with at least one agent selected from the group consisting of PACAP, DHEA and LiCl.

In exemplary embodiments, the MSCs of the methods and compositions of the invention are bone-marrow derived MSCs. In another embodiment, the MSCs are mammalian cells. In yet another embodiment, the MSCs are human cells.

According to some embodiments, said subject is a healthy subject. According to another embodiment, said subject is a subject suffering from a neurodegenerative or neuropsychiatric disease. In one embodiment, said subject is a subject suffering from a neurodegenerative disease. In another embodiment, the neurodegenerative disease is Alzheimer's disease. In another embodiment, said subject is a subject suffering from a neuropsychiatric disease. According to another embodiment, the neuropsychiatric disease is schizophrenia.

According to another embodiment, the methods and compositions of the invention have a long term effect in improving the cognitive function of said subject. According to a particular embodiment, the long term effect is for at least 2 months following administration. According to yet another particular embodiment, the long term effect is for at least 3 months following administration. As used herein, the term “following administration” relates to either a single administration or to sequential administration.

According to additional embodiments of the methods of the present invention, said MSCs administration is intracerebroventricular administration. According to other embodiments, said MSCs administration is intravenous administration.

According to another aspect, the present invention provides a method for generating MSCs comprising culturing MSCs in the presence of an effective amount of at least one agent selected from the group consisting of PACAP a fragment or analog thereof, LiCl and DHEA. In particular embodiments, the at least one agent is PACAP. In yet another particular embodiment, the at least one agent is a PACAP-27. In one embodiment, the methods enhance the neurogenic activity of said MSCs.

In another embodiment, the neurogenic MSCs are useful for treating a neurodegenerative or neuropsychiatric disease. In another embodiment, the neurogenic MSCs are useful for improving cognitive function of a subject. In another embodiment, said method is useful for adapting said MSCs for systemic administration. In a particular embodiment, the systemic administration is intravenous administration.

According to another aspect, the present invention provides a method of treating schizophrenia in a subject in need thereof, comprising the steps of administering to said subject a pharmaceutically effective amount of isolated MSCs. According to some embodiments, the method improves at least one of cognitive function or affective behavior in said subject. In one embodiment, said treatment comprises improvement of a cognitive function in said subject. In another embodiment, said treatment comprises improvement of an affective behavior in said subject.

According to some embodiments, said method comprises pretreatment of the MSCs with an effective amount of PACAP or analogs or fragments thereof. According to another embodiment, said method comprises pretreatment of the MSCs with an effective amount of PACAP-27.

According to another aspect, there is provided cell based compositions comprising isolated MSCs for use in treating a disease selected from the group consisting of a neurodegenerative disease and a psychiatric disease. According to another aspect, there is provides use of cell based compositions comprising isolated MSCs for preparation of a medicament for treating a neurodegenerative disease or a psychiatric disease in a subject in need thereof. In some embodiments, the psychiatric disease comprises an affective disorder. According to particular embodiments, said psychiatric disease is schizophrenia. According to some embodiment, said treatment comprises improvement of at least one of cognitive function and affective behavior in said subject. According to particular embodiments, said neurodegenerative disease is Alzheimer's disease.

According to another aspect, there is provided an isolated MSC cultured in the presence of an effective amount of an agent selected from the group consisting of PACAP, a PACAP analog, a PACAP fragment, DHEA and LiCl, the MSC having enhanced neurogenic activity.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

Other objects, features and advantages of the present invention will become clear from the following description and drawings.

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

### BRIEF DESCRIPTION OF THE DRAWINGS

FIG. 1 presents an immunohistochemical stain of a neurosphere stained with neuronal and glial markers nestin; doublecortin (DCX); and glial fibrillary acidic protein (GFAP);

FIG. 2 is a bar graph representing the number of developing neurospheres in vitro in various media;

FIG. 3 is a line graph representing the effect of intracerebroventricular injection of MSCs and activated MSCs on cognitive function (spatial learning in the Morris Water Maze MWM test) in normal mice;

FIG. 4 is a line graph representing the effect of intravenous injection of MSCs and activated MSCs on cognitive function (MWM) in normal mice;

FIG. 5 is a picture showing the presence of fluorescently labeled MSCs in the dentate gyrus;

FIG. 6 is a bar graph representing the effect of MSCs and PACAP-activated MSCs on neurogenesis in normal mice;

FIG. 7 is an immunohistochemical stain showing the presence of increased microglial cells in the dentate gyrus of mice treated with PACAP-activated MSCs in normal mice;

FIG. 8 is a line graph representing the effect of MSCs on cognitive function (MWM) in a murine model of acute Alzheimer's disease;

FIG. 9 is a bar graph representing the effect of MSCs treatment at adulthood on social discrimination in ketamine-injected pups as a murine model of schizophrenia;

FIG. 10 is a bar graph representing the effect of MSCs treatment at adulthood on pre-pulse inhibition (PPI) in ketamine-injected pups as a murine model of schizophrenia;

FIG. 11 is a bar graph representing the effect of MSC treatment at adulthood on acoustic startle response in ketamine-injected pups as a murine model of schizophrenia;

FIG. 12 is a line graph representing the effect of MSC treatment at adulthood on cognitive function (spatial learning in the Morris Water Maze test) in pups injected with ketamine at day 10 only;

FIG. 13 is a bar graph representing the effect of MSC treatment in adulthood on neurogenesis in the dentate gyrus of ketamine-treated pups; and

FIG. 14 is a bar graph representing the effect of MSC treatment on PPI in ketamine-injected adult male mice 15 minutes before testing;

FIG. 15A-15C are bar graphs representing the short term effect (2 weeks) of MSC and clozapine (10 days) treatments in adulthood on PPI in a murine model of schizophrenia (ketamine-injected female mice pups), with varying pre-pulse intensity: 70 db, 74 db and 82 db, respectively (*P<0.05 vs. Ket control);

FIG. 16A-16C are bar graphs representing the long term effect (2 months) of MSC treatment and clozapine (6 weeks after treatment) in adulthood on PPI in a murine model of schizophrenia (ketamine-injected female mice pups), with varying pre-pulse intensity: 70 db, 74 db and 82 db, respectively (*P<0.05 vs. Ket control);

FIG. 17A-17C are bar graphs representing the long term effect (3 months) of MSC treatment and clozapine (2.5 months after treatment) in adulthood on PPI in a murine model of schizophrenia (ketamine-injected female mice pups), with varying pre-pulse intensity: 70 db, 74 db and 82 db, respectively. (FIG. 17A: *P<0.05 vs. Ket control; FIG. 17B: *P<0.05 vs. Ket control and Ket+MSC (iv); FIG. 17C: *P<0.05 vs. Ket control, #P<0.05 vs. clozapine);

FIG. 18 is a bar graph representing the effect of MSC treatment on social recognition test in a murine model of schizophrenia (ketamine-injected female mice pups). (*P<0.05 vs. Ket control & Ket+MSCp(icv), #P<0.05 vs. clozapine, $P<0.05 vs. Ket+MSC(icv) & saline); and

FIG. 19 is a bar graph representing the long term effect (3 months) of MSC treatment on social recognition test in a murine model of schizophrenia (ketamine-injected female mice pups). (*P<0.05 vs. Ket control, Ket+MSCp(icv) & clozapine).

### DETAILED DESCRIPTION OF THE INVENTION

The present invention relates to the field of stem cell therapy and more particularly, but not exclusively, to the use of mesenchymal stem cells for the treatment of neurodegenerative, neurobehavioral or psychiatric disorders, and for improvement of affective and cognitive behavior.

In some embodiments, the improvement of affective and cognitive behavior is achieved in a normal subject. In some embodiments, the improvement of affective and cognitive behavior is achieved in a subject suffering from a neurodegenerative or neuropsychiatric disease, such as, for example, Alzheimer's disease or schizophrenia.

As exemplified herein below, incubation of MSCs in culture with at least one of pituitary adenylate cyclase-activating polypeptide (PACAP), dehydroepiandrosterone (DHEA) or lithium chloride (LiCl) enhanced the neurogenic activity of the MSCs, in vitro. Further, transplantation of bone marrow-derived MSCs via direct injection into the lateral ventricle improved cognitive functions in normal mice (spatial learning). “Activated” MSCs, i.e., MSCs pre-treated with PACAP-27, surprisingly exhibited a significant effect on cognitive behavior in normal mice also when administered intravenously and not directly to the brain.

Direct injection of MSCs into the lateral ventricle in the brain was found to result in improved spatial learning (cognitive function) in a murine model of acute Alzheimer's disease induced by amyloid beta aggregates injection into the lateral ventricle.

Direct injection of MSCs into the lateral ventricle was found to improve spatial learning, social preference and pre-pulse inhibition in murine models of schizophrenia induced by the N-methyl D-aspartate (NMDA) antagonist ketamine. The models used were a developmental model wherein ketamine was injected into pups, which were treated with MSCs at adulthood after which behavioral tests conducted; and an acute model, wherein a high dose of ketamin was injected into normal mice previously injected with MSCs or their sham controls 15 minutes before testing for pre-pulse inhibition (PPI). Surprisingly, the MSCs of the invention exhibited long term improvement in social preference and pre-pulse inhibition in murine model of schizophrenia.

According to some embodiments of the present invention there is provided a method of generating MSCs having enhanced neurogenic activity, the method comprising incubating MSCs in a culture medium comprising at least one agent selected from PACAP or a fragment or analog thereof, DHEA or LiCl; thereby generating cells MSCs having enhanced neurogenic activity. Advantageously, said cell are useful for treating psychiatric disorders (including but not limited to schizophrenia), neurodegenerative diseases (including but not limited to Alzheimer's disease) and for improving cognitive and affective function of a subject in need thereof.

As used herein, “PACAP” refers to Pituitary Adenylate Cyclase-Activating Polypeptide (also known as adenylate cyclase-activating polypeptide 1; ADCYAP1) and refers to the mature PACAP as well as processed versions of the mature PACAP. The mature form of human PACAP consists of 176 amino as set forth in SEQ ID NO: 9 (MTMCSGARLALLVYGIIMHS SVYSSPAAAGLRFPGIRPEEEAYGEDGNPLPDFDGSEPPGAGSPASAPRAA AAWYRPAGRRDVAHGILNEAYRKVLDQLSAGKHLQSLVARGVGGSLGGGA GDDAEPLSKRHSDGIFTDSYSRYRKQMAVKKYLAAVLGKRYKQRVKNKGR RIAYL; Accession No. NP_{—}001093203.1).

In some embodiment, the PACAP peptide of the invention is a fragment of 5-60, 10-50, 15-45, 20-50 amino acid derived from SEQ ID NO: 9. In some embodiments, the PACAP peptide comprises at least 5, 10, 15, 16, 17, 18, 19, 20, 21, 23, 24, 25, 26 or 27 amino acids derived from SEQ ID NO: 9. In additional embodiments, the PACAP peptide comprises no more than 60, 55, 50, 45, 44, 43, 42, 41, 40, 39, 38, 37, 36, 35, 34, 33, 32, 31, 30, 29 or 28 amino acids derived from SEQ ID NO: 9. In another embodiment, the PACAP peptide is derived, or corresponds to, amino acids 132-158 of SEQ ID NO: 9, or a fragment thereof. In yet another embodiment, the PACAP peptide is derived, or corresponds to, amino acids 132-169 of SEQ ID NO: 9, or a fragment thereof.

In another embodiment, the PACAP has the amino acid sequence as set forth in SEQ ID NO: 1 (HSDGIFTDSYSRYRKQMAVKKYLAAVL).

In another embodiment, the PACAP has the amino acid sequence as set forth in SEQ ID NO: 2 (HSDGIFTDSYSRYRKQMAVKKYLAAVLGKRYKQRVKNK).

In another embodiment, the PACAP has the amino acid sequence as set forth in SEQ ID NO: 3 (FTDSYSRYRKQMAVKKYLAAVL).

In another embodiment, the PACAP has the amino acid sequence as set forth in SEQ ID NO: (FTDSYSRYRKQMAVKKYLAAVLGKRYKQRVKNK).

In another embodiment, the present invention further encompasses PACAP homologs or fragments thereof, as agents capable of enhancing MSCs neurogenic activity. In another embodiment, the present invention further encompasses ligand molecules similar to PACAP, i.e., ligands who bind the same receptor as PACAP. For instance, it is known in the art that PACAP and vasoactive intestinal peptide (VIP) are structurally related. In some embodiments, the PACAP homolog is VIP (the prepro-vasoactive intestinal peptide may have the GenBank accession no.: AAA61289.1 or AAA61284.1). In another embodiment the agent is a VIP peptide or an analog or fragment, thereof. In another embodiment the VIP is selected from the group consisting of:

SEQ ID NO: 5
(HSDAVFTDNYTRLRKQMAVKKYLNSILN),
SEQ ID NO: 6
(HSDAVFTENYTKLRKQLAAKKYLDLKKGGT),
SEQ ID NO: 7
(HSDAVFTENYTKLRKQLAAKKYLDLKK)
and
SEQ ID NO: 8
(HSDAVFTNSYRKVLKRLSARKLLQDIL).

In another embodiment, the VIP agent may be cyclized, such as Ro 25-1553 (SEQ ID NO: 10; Ac-HSDAVFTENYTKLRKQLAAKKYLDLKKGGT) or Ro 25-1392 (SEQ ID NO: 11; Ac-HSDAVFTENYTKLRKQLAAKKYLDLKK), known in the art as cyclic peptide analogs of VIP.

The terms “polypeptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to aspartic acid (D). Conservative substitution tables providing functionally similar amino acids are well known in the art.

The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) (see, e.g., Creighton, Proteins, 1984).

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

The phrase “conservative substitution” also includes the use of a chemically derivatized residue in place of a non-derivatized residue provided that such peptide displays the requisite function of as specified herein.

The term “derived from” or “corresponding to” refers to construction of a peptide based on the knowledge of a sequence using any one of the suitable means known to one skilled in the art, e.g. chemical synthesis in accordance with standard protocols in the art.

The PACAP peptides of the invention may be synthesized or prepared by techniques well known in the art. The peptides can be synthesized by a solid phase peptide synthesis method of Merrifield (see J. Am. Chem. Soc., 85:2149, 1964). Alternatively, the peptides of the present invention can be synthesized using standard solution methods well known in the art (see, for example, Bodanszky, M., Principles of Peptide Synthesis, Springer-Verlag, 1984) or by any other method known in the art for peptide synthesis.

In additional embodiments, the at least one agent useful in enhancing MSCs neurogenic activity is Dehydro-epi-androsterone (DHEA; CAS Number: 53-43-0). DHEA is a 17-ketosteroid which is quantitatively one of the major adrenocortical steroid hormones found in mammals. DHEA may be commercially obtained through various sources, e.g. Sigma Aldrich.

In additional embodiments, the at least one agent useful in enhancing MSCs neurogenic activity is lithium chloride (LiCl; CAS Registry Number: 7447-41-8). LiCl may be commercially obtained through various sources.

Mesenchymal Stem Cells

The term “mesenchymal stem cell” or “MSC” is used interchangeably for cells which are not terminally differentiated, which can divide without limit, to yield cells that are either stem cells, or which, irreversibly differentiate into specific mesenchymal tissues lineages, including bone, cartilage, fat, tendon, muscle and bone marrow stroma. Various markers have been described on mesenchymal stem cells including CD13, CD29, CD44, CD90, CD105, SH-3, and STRO-I. In another embodiment, the MSC are negative for CD34, CD45, CD11b markers.

In some aspects the MSCs are isolated from a group selected of: bone marrow, adipose tissue, umbilical cord blood, placental tissue, peripheral blood mononuclear cells, gingival tissue, differentiated embryonic stem cells, and differentiated progenitor cells. In one embodiment, the MSCs of the present invention are adult cells. In another embodiment, the MSCs of the present invention are human.

In an exemplary embodiment, the MSCs are bone marrow derived MSCs. Bone marrow cells may be obtained from iliac crest, femora, tibiae, sternum, spine, rib or other medullary spaces.

Bone marrow stromal cells may be commercially obtained through various sources. For example, bone-marrow derived MSCs isolated from human, mouse, rat, rabbit, dog, goat, sheep, pig, and horse are available from Cognate Bioservices Incorporated (Baltimore, Md.) Lonza Group Ltd., Osiris Therapeutics Inc. and Mesoblast Ltd.

Alternatively, the cells may be freshly isolated from any animal, by methods well known to those of ordinary skill in the art. In some embodiments, the cells are derived from mammals, and in particular embodiments, the stromal cells are derived from humans.

The term “isolated” as used herein refers to a cell that has been removed from its in-vivo location (e.g. bone marrow). Preferably the isolated cell is substantially free from other substances (e.g., other cell types) that are present in its in-vivo location.

In any of the methods described herein the cells may be obtained from any autologous or non-autologous (i.e., allogeneic or xenogeneic) human donor. In one embodiment, the MSCs are of an autologous source. In another embodiment, the MSCs are of an allogeneic source. In another embodiment, the MSCs are of syngeneic source. In yet another embodiment, the MSCs are of xenogeneic source.

Sources of bone-marrow derived MSCs and methods of obtaining and culturing said cells from those sources have been described in the art (e.g., Friedenstein et al., 1976 Exp. Hematol. 4: 267-274; Friedenstein et al., 1987, Cell Tissue Kinetics 20: 263-272; Castro-Malaspina et al., 1980, Blood 56: 289-301; Mets et al., 1981, Mech. Aging Develop. 16: 81-89; Piersma et al., 1985, Exp. Hematol. 13: 237-243; Owen et al., 1988, Cell and Molecular Biology of Vertebrate Hard Tissues, Ciba Foundation Symposium, Chichester, U. K., 42-60; Caplan, 1991, J. Orthoped. Res. 9: 641-650; Prockop, 1997, Science 276: 71-74; Beresford et al., 1992, J. Cell Sci. 102: 341-351; Cheng et al., 1994, Endocrinology 134: 277-286; Rickard et al., 1994, Develop. Biol. 161: 218-228; Clark et al., 1995, Ann. N.Y. Acad. Sci. 770:70-78). Bone-marrow derived MSCs can be obtained from substantially any bone marrow including, for example, bone marrow obtained by aspiration of the iliac crest of human donors. Methods for obtaining bone marrow from donors are well known in the art.

Following isolation, the cells are typically expanded by culturing in a proliferation medium capable of maintaining and/or expanding the isolated cells ex vivo in the presence of platelet lysate. The proliferation medium may be DMEM, alpha-MEM or DMEM/F12. It will be appreciated that preferably when the MSCs are human, the platelet lysate is also obtained from human cells. According to one embodiment, the medium is devoid of xeno contaminants i.e. free of animal derived components. According to another embodiment, the medium comprises components derived from the serum of the subject the MSC have been derived from. According to another embodiment, the MSC are cultures in serum obtained from the subject. According to yet another embodiment, the medium may be commercially obtained through various sources, such as providers of MSCs as described herein.

The MSCs may be cultured in growth-promoting conditions, which can include any set of conditions (temperature, atmosphere, growth medium composition, humidity, degree of agitation, etc.) under which the cells normally proliferate. The temperature should be near that of normal human body temperature (i.e., about 37° C.), but can be any temperature at which stromal cells can proliferate (e.g., 30 to 43° C.). Stromal cells can be grown in an air atmosphere, or an air atmosphere supplemented with 5-10% CO₂, for example. The growth medium can be any liquid medium which contains nutrients and factors sufficient to support proliferation of MSC cells. Such media contain, for example, a carbon source (e.g., glucose) and minimal essential nutrients, and preferably contain one or more of a mammalian serum (e.g., fetal calf serum), an antibiotic (e.g., penicillin or streptomycin), and L-glutamine (i.e., to improve amino acid supply for protein biosynthesis). The growth medium may further contain vitamins, amino acids and growth factors including but not limited to EGF and bFGF.

The mammalian serum can be used at a concentration of 1% to 20%, by volume, of the total growth medium. The serum is preferably pre-screened to ensure that it supports vigorous growth of stromal cells; some lots, even lots provided from the same supplier, do not support vigorous growth of stromal cells. Alternatively, the mammalian serum can be replaced with one or more growth factors (e.g., fibroblast growth factor, platelet derived growth factor, insulin growth factor, or endothelial growth factor). The growth medium can, for example, be Minimal Essential Medium-alpha without deoxyribonucleotides or ribonucleotides, supplemented with fetal calf serum, antibiotics, and L-glutamine; Dulbecco's minimal essential medium; and others well known to one of ordinary skill in the art. The growth medium is preferably replaced one or more times (e.g., every 3 or 4 days) during culture of the stromal cells.

One of ordinary skill in the art would appreciate, for example, that MSCs can be expanded and simultaneously retain a pluripotent state (i.e., the ability to differentiate into one of numerous cells types, such as osteoblasts, adipocytes, and cells of the CNS, for example). Moreover, methods to differentiate MSCs into various cell types in vitro have been described in the art (e.g., WO 96/30031, WO 99/43286, and U.S. Pat. No. 7,279,331).

MSC cells of the methods of the present invention can be cultured using art-known methods for a period of about 1 hour to 1 year. Typically, following isolation of the MSCs from the source tissue, the cells are expanded for about 30 days (about 6-8 passages). The cells can be frozen and thawed, thereafter maintained in culture for about 1 to 30 days, about 5 to 20 days, or about 3 to 14 days and are preferably harvested after not more than about 14 days, 10 days, or 7 days.

MSC cells can be expanded by seeding the cells on a growth surface in the presence of a growth medium, and then harvesting the cells after, e.g., 15-30 days). Alternatively, the stromal cell expansion can be performed in series, meaning that the cells are expanded more than once. For example, after a first expansion on a first growth surface, stromal cells are harvested and then expanded in a growth medium on a second growth surface. Of course, the twice-expanded stromal cells can be harvested and subjected to one or more additional rounds of expansion, using the same method.

Additionally, one of ordinary skill in the art would recognize that methods for isolating the different types of stromal cells described herein are known in the art (e.g., ADSC's, Rodbell (1964) J Biol Chem 239:375 and Hanuer et al. (1989) J Clin Invest 84:1663-1670; LSCs, U.S. Patent App. Pub No. 2006/0057125; and Wharton's jelly stromal cells, McElreavey et al., 1991, Biochem. Soc. Trans. 636.sup.th Meeting Dublin 19:29 S and U.S. Patent App. Pub No. 2004/0136967).

The cells of the present invention can be administered to the treated individual using a variety of transplantation approaches, the nature of which depends on the site of implantation. Alternatively, the cells of the present invention may be administered systemically, including but not limited to, intravenous administration.

The term or phrase “transplantation” or “grafting” are used interchangeably herein and refer to the introduction of the cells of the present invention to target tissue. The cells of the invention can be directly transplanted by intracerebroventricular, intraparenchymal, intraspinal, intracisternal or intracranial administration.

The cells can be grafted into the central nervous system or into the ventricular cavities or subdurally onto the surface of a host brain. Intraparenchymal transplantation can be effected using two approaches: (i) injection of cells into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells. For subdural grafting, the cells may be injected around the surface of the brain after making a slit in the dura. Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the cell suspension is drawn up into the syringe and administered to anesthetized transplantation recipients. Multiple injections may be made using this procedure.

The cellular suspension procedure thus permits grafting of the cells to any predetermined site in the brain, is relatively non-traumatic, allows multiple grafting simultaneously in several different sites or the same site using the same cell suspension, and permits mixtures of cells from different anatomical regions. Multiple grafts may consist of a mixture of cell types, and/or a mixture of transgenes inserted into the cells. Preferably from approximately 10⁴ to approximately 10¹⁰ cells are introduced per graft.

For transplantation into cavities, tissue is removed from regions close to the external surface of the central nerve system (CNS) to form a transplantation cavity, for example as described by Stenevi et al. (Brain Res. 114:1-20., 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated and the astrocytic phenotype comprised in the cell (e.g. particular neurotrophic factor being secreted) by the cells of the present invention.

Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation. Recent publications indicate that MSCs have immunosuppressive properties and may avoid the use of additional immunosuppression (Ucceli A et al. 2008).

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64). Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J. Microencapsul. 2000, 17: 245-51.

Cell-Based Compositions

As used herein, a “pharmaceutical composition” refers to a preparation of one or more of the active ingredients described herein, with other components such as physiologically suitable carriers and excipients. The purpose of a pharmaceutical composition is to facilitate administration of a compound to a subject.

Hereinafter, the phrases “therapeutically acceptable carrier” and “pharmaceutically acceptable carrier”, which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

In another embodiment of the present invention, a therapeutic composition further comprises a pharmaceutically acceptable carrier. As used herein, a “carrier” refers to any substance suitable as a vehicle for delivering of the agents or molecule of the present invention to a suitable in vivo or in vitro site. As such, carriers can act as a pharmaceutically acceptable excipient of a therapeutic composition of the present invention. Carriers of the present invention include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Therapeutic compositions of the present invention can be sterilized by conventional methods.

The term “effective amount” or as used herein refers to the amount of active ingredient or active component in a pharmaceutical composition that will achieve the desired goal, e.g. enhancing the neurogenic activity of MSCs useful for improving cognitive function of a subject.

In some embodiments, the pretreatment (e.g. incubation of MSCs with an agent such as PACAP) is for about 1 to about 21 days. In additional embodiments, the pretreatment is for 1 to 7 days. In another embodiment, the pretreatment is for 3 to 7 days. In additional embodiments, the treatment is a daily treatment. In additional embodiments, the treatment is a daily treatment with 2-3 days intervals. In some embodiments the MSCs are treated with 1 nM to 500 nM PACAP. Effective doses can be extrapolated from dose-response curves derived from in-vitro or in-vivo animal model test bioassays or systems

The amount of the MSCs of the present invention, which will be effective in the treatment of a particular disorder or condition will depend on the nature of the disorder or condition and can be determined by standard clinical techniques known to a person skilled in the art. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the effective amount of MSCs when administered intracerebroventricularly is 10⁴-10¹⁰. In another embodiment, the effective amount of MSCs when administered intravenously is 10⁶-10¹².

Cognitive Function

According to some embodiments of the present invention, the cell based compositions of the present invention are useful for improving cognitive function of an individual. In some embodiments, the improving cognitive function is achieved in healthy subjects. In other embodiments, the improving cognitive function is achieved in subjects suffering from cognitive dysfunctions. In another embodiment, improving cognitive function includes promoting cognitive function and/or preserving cognitive function in a subject.

The term “cognitive function” as used herein, refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care.

In humans, cognitive function may be measured by means known in the art, for example and without limitation, by the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test (Buschke and Fuld, 1974); the Verbal Paired Associates subtest; the Logical Memory subtest; the Visual Reproduction subtest of the Wechsler Memory Scale-Revised (WMS-R) (Wechsler, 1997); the Benton Visual Retention Test, or the explicit 3-alternative forced choice task. See Folstein et al., J Psychiatric Res 12: 189-98, (1975); Robbins et al., Dementia 5: 266-81, (1994); Rey, L'examen clinique en psychologie, (1964); Kluger et al., J Geriatr Psychiatry Neurol 12:168-79, (1999); Marquis et al., 2002 and Masur et al., 1994.

In animal model systems, cognitive function may be measured in various conventional ways known in the art, including using a Morris Water Maze (MWM) (as exemplified herein-below), Barnes circular maze, elevated radial arm maze, T maze or any other mazes in which the animals use spatial information. Other tests known in the art may also be used to assess cognitive function, such as novel object recognition and odor recognition tasks.

Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

In another embodiment, the invention provides methods for preserving cognitive function. In some embodiments, preserving cognitive function is affecting normal or impaired cognitive function such that it does not decline or does not fall below that observed in the subject upon first presentation or diagnosis, or delays such decline.

Typically, treating a disorder of cognitive function according to the present invention includes treating, controlling, preventing and/or reducing one or more clinical signs (i.e., symptoms) of cognitive impairment in a subject in need thereof. These impairments can result from disorders such as age-associated memory dysfunction, memory loss, mild cognitive impairment, cognitive dysfunction syndrome, and dementias. Such dementias include, but are not limited to, Alzheimer's disease, Lewy body dementia, vascular dementia, dementia caused by chronic cerebral ischemia, AIDS dementia, dementia caused by Parkinson's disease, dementia caused by amyotrophic lateral sclerosis, dementia caused by brain trauma, dementia caused by Huntigton's disease, dementia caused by multiple sclerosis, dementia caused by Pick's disease, dementia caused by vascular disease, dementia caused by organ system failure, dementia caused by metabolic diseases, and dementia caused by infectious. Generally recognized compendiums of disorders that accompanied with decline of cognitive functions are Merck Manual of Diagnosis and Therapy. Sect. 14 Neurologic Disorders, Chapt. 171. Merck Manual of Geriatrics Sect.5, Chapt. 40.

In some embodiments, promoting cognitive function is affecting impaired cognitive function so that it more closely resembles the function of a normal, unimpaired subject. Cognitive function may be promoted to any detectable degree, but in humans preferably is promoted sufficiently to allow an impaired subject to carry out daily activities of normal life at the same level of proficiency as a normal, unimpaired subject.

As used herein, the term “impaired cognitive function”, “cognitive impairment” or “cognitive dysfunction” as used herein refers to cognitive function in subjects that is not as robust as that expected in a normal, unimpaired subject. In some cases, cognitive function is reduced by about 5%, about 10%, about 30%, or more, compared to cognitive function expected in a normal, unimpaired subject. In some cases, “cognitive impairment” or “cognitive dysfunction” in subjects affected by aged-related cognitive impairment refers to cognitive function in subjects that is not as robust as that expected in an aged-matched normal, unimpaired subject, or the function of a young adult subject (i.e. subjects with mean scores for a given age in a cognitive test).

In another embodiment, the present invention provides methods for promoting or enhancing cognitive function in a subject affected by age-related cognitive. Typically, promoting cognitive function in a subject affected by age-related cognitive refers to affecting impaired cognitive function so that it more closely resembles the function of an aged-matched normal, unimpaired subject, or the function of a young adult subject.

“Age-related cognitive impairment” refers to cognitive impairment in aged subjects, wherein their cognitive function is not as robust as that expected in an age-matched normal subject or as that expected in young adult subjects. In some cases, cognitive function is reduced by about 5%, about 10%, about 30%, or more, compared to cognitive function expected in an age-matched normal subject. In some cases, cognitive function is as expected in an age-matched normal subject, but reduced by about 5%, about 10%, about 30%, about 50% or more, compared to cognitive function expected in a young adult subject. Age-related impaired cognitive function may be associated with Mild Cognitive Impairment (MCI) (including amestic MCI and non-amnestic MCI), Age-Associated Memory Impairment (AAMI), and Age-related Cognitive Decline (ARCD).

“Mild Cognitive Impairment” or “MCI” refers to a condition characterized by isolated memory impairment unaccompanied other cognitive abnormalities and relatively normal functional abilities. One set of criteria for a clinical characterization of MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines).

“Age-Associate Memory Impairment (AAMI)” refers to a decline in memory due to aging. A patient may be considered to have AAMI if he or she is at least 50 years old and meets all of the following criteria: a) The patient has noticed a decline in memory performance, b) The patient performs worse on a standard test of memory compared to young adults, c) All other obvious causes of memory decline, except normal aging, have been ruled out (in other words, the memory decline cannot be attributed to other causes such as a recent heart attack or head injury, depression, adverse reactions to medication, Alzheimer's disease, etc.).

“Age-Related Cognitive Decline (ARCD)” refers to declines in memory and cognitive abilities that are a normal consequence of aging in humans.

Alzheimer's disease (AD) is characterized by memory deficits in its early phase. Later symptoms include impaired judgment, disorientation, confusion, behavior changes, trouble speaking, and motor deficits. Histologically, AD is characterized by beta-amyloid plaques and tangles of protein tau.

In another embodiment, the cell based compositions of the present invention are useful for enhanced learning of an individual such as for restoring learning capability after a decline in a learning capability. In another embodiment, the term learning includes memory, memory storage, training capability and/or capacity, and the ability to study. In another embodiment, enhancing learning is enhancing spatial and/or non-spatial learning.

In another embodiment, the term enhancing is interchangeable with the terms “promoting”, “intensifying”, “improving”, “increasing”, “inducing”, and “expanding”.

In another embodiment, learning comprises memory or memorization. In another embodiment, increase or enhancement in memory or memorization comprises changes in strength of connections between neurons in the relevant networks underling memory storage. In another embodiment, increase or enhancement in memory or memorization comprises modifications in intrinsic neuronal properties. In another embodiment, increase or enhancement in learning as described herein comprises behavioral changes.

In another embodiment, enhancing learning is enhancing sensory memory. In another embodiment, enhancing learning is enhancing short-term memory. In another embodiment, enhancing learning is enhancing long-term memory. In another embodiment, enhancing learning is enhancing memorization. In another embodiment, enhancing learning is enhancing declarative memory or explicit memory. In another embodiment, enhancing learning is enhancing implicit memory.

In another embodiment, enhancing learning is enhancing learning ability. In another embodiment, enhancing learning is enhancing mental capacity. In another embodiment, enhancing learning results in intelligence enhancement. In another embodiment, enhancing learning is attaining higher rates of learning without unacceptable reduction of comprehension or retention.

According to some embodiments of the present invention, the cell based compositions of the present invention are useful for treating a disorder, or condition of the central nervous system. Neurological condition to be treated by the MSCs of the invention and pharmaceutical compositions comprising same, include neuronal cell death following acute insults such as hypoxia, ischemia, stroke, and trauma. Other neurological conditions treatable with agent of the invention include Alzheimer's disease, AIDS dementia, epilepsy, focal ischemia, Huntington's disease, Parkinson's disease, and amyotrophic lateral sclerosis. Each of these conditions is characterized by the progressive loss of a specific population of neurons in the central nervous system.

According to some embodiments of the present invention, the cell based compositions of the present invention are useful for treating a disease or disorder associated with reduced or impaired neurogenesis including but not limited to neurogenesis in the hippocampus.

According to some embodiments of the present invention, the cell based compositions of the present invention are useful for treating neurodevelopmental impairment caused by exposure to toxins or pre-natal and post-natal stress.

The term “schizophrenia” as used herein refers to a neuropsychiatric disorder in which the patient suffers from distorted thinking, hallucinations, and a reduced ability to feel normal emotions. The term “positive symptoms” as used herein refers to the presence of distinctive behaviors in a schizophrenic patient, such as, but not limited to, strange or paranoid delusions, hallucinations, and fearful reaction to ordinary sights. The term “negative symptoms” as used herein refers to the absence of normal social and interpersonal behaviors in a schizophrenic patient. The current anti-psychotics do not treat the negative symptoms of Schizophrenia but only the positive symptoms. The methods and compositions of the present invention provide a means to treat both negative and positive symptoms for long terms.

A major key phenotype tested in autism spectrum disorders (ASD) is the social preference test shared with Schizophrenia models, as tested herein. According to some embodiments, the compositions and methods of the invention are useful in treating autism spectrum disorders According to other embodiments, the compositions and methods of the invention are useful in treating ADHD and attention impairment.

According to other embodiments, the compositions and methods of the invention are useful in treating depression. In particular embodiments, activated MSCs (including MSC pretreated with PACAP) are useful in treating depression.

According to some embodiments, said subject is a healthy (e.g. normal) subject. In another embodiment, a healthy subject is subject not afflicted with a degenerative brain disorder. In another embodiment, a healthy subject is subject not afflicted with cognitive dysfunction. In another embodiment, a healthy subject is subject not afflicted with a neuropsychiatric disease. In another embodiment, a healthy subject is subject not afflicted with depression.

In another embodiment, a subject in need of a treatment according to a method such as described herein is afflicted with a cognitive and/or degenerative brain disorder. In another embodiment, a subject in need of a treatment according to a method such as described herein is suffering from memory loss. In another embodiment, a subject in need of a treatment according to a method such as described herein is suffering from a progressive loss of memory, cognition, reasoning, judgment, emotional stability, or any combination thereof.

In another embodiment, a subject in need of a treatment according to a method such as described herein is suffering from Alzheimer's disease (AD). In another embodiment, a subject in need of a treatment according to a method such as described herein is suffering from dementia. In another embodiment, a subject in need of a treatment according to a method such as described herein is suffering from multi-infarct dementia. In another embodiment, a subject in need of a treatment according to a method such as described herein is suffering from mixed organic brain syndrome metabolic encephalopathies of various origins. In another embodiment, a subject in need of a treatment according to a method such as described herein is suffering from alcoholic dementia. In another embodiment, a subject in need of a treatment according to a method such as described herein is suffering from a learning disorder. In another embodiment, a subject in need of a treatment according to a method such as described herein is suffering from loss of learning and memory associated with neuronal damage.

In another embodiment, a subject in need of a treatment according to a method such as described herein is suffering from a learning disability caused by a non-degenerative disorder. In another embodiment, a subject in need of a treatment according to a method such as described herein is suffering from a cognitive impairment. In another embodiment, a subject in need of a treatment according to a method such as described herein is suffering from an age-related cognitive decline. In another embodiment, a subject in need of a treatment according to a method such as described herein is suffering from a cerebral senility. In another embodiment, a subject in need of a treatment according to a method such as described herein is suffering from vascular dementia. In another embodiment, a subject in need of a treatment according to a method such as described herein is suffering from AIDS-associated dementia. In another embodiment, a subject in need of a treatment according to a method such as described herein is suffering from electric shock induced amnesia. In another embodiment, a subject in need of a treatment according to a method such as described herein is suffering from Parkinson's disease. In another embodiment, a subject in need of a treatment according to a method such as described herein is suffering from Down's syndrome. In another embodiment, a subject in need of a treatment according to a method such as described herein is suffering from a metal retardation including but not limited to fragile X syndrome. In another embodiment, a subject in need of a treatment according to a method such as described herein is suffering from stroke. In another embodiment, a subject in need of a treatment according to a method such as described herein is suffering from traumatic brain injury. In another embodiment, a subject in need of a treatment according to a method such as described herein is suffering from Huntington's disease. In another embodiment, a subject in need of a treatment according to a method such as described herein is suffering from an attention deficit disorder and/or hyperactivity disorders.

In another embodiment, a subject in need of a treatment according to a method such as described herein is a healthy subject in need of improved cognitive function. In another embodiment, the subject is a human subject. In another embodiment, the subject is selected from a farm animal, a rescue animal (e.g., a dog).

The principles, uses and implementations of the teachings herein may be better understood with reference to the accompanying description and figures. Upon perusal of the description and figures present herein, one skilled in the art is able to implement the invention without undue effort or experimentation.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

Exemplary embodiments are discussed herein below with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

### EXPERIMENTAL

### Mesenchymal Stem Cell Isolation and Expansion In Vitro

Mesenchymal stem cells were isolated from the bone marrow of female SABRA and ICR mice. Briefly, following the sacrifice of the mice, the tibias and femurs were removed and cleaned of connective tissue. Marrow was flushed out of the cut bones after removal of the epiphysis and suspended in Dulbecco's modified Eagle's medium (DMEM; Biological Industries, Beit Haemek, Ill.) supplemented with 20% fetal bovine serum (FBS; Biological Industries), 100 units/ml penicillin (Biological Industries), 100 μg/mL streptomycin (Biological Industries), 4 mM L-glutamine (Biological Industries), vitamin solution (Biological Industries) and non-essential amino acids (Biological Industries 1:100). Marrow cells were separated and suspended by repeated passage through 19G, 20G, 21G, 23G and 25G syringe needles. Suspended marrow cells (10⁸) were plated in 100 mm² dishes and cultured under conditions of 37° C. and 10% CO₂. The non-adherent cells were removed 24 and 48 h after plating. MSCs were expanded in culture for 1-3 passages, after which expansion medium with a reduced serum content (10% FBS) was used. The medium was changed twice weekly. MSCs were frozen in a freezing cocktail containing the expansion medium with 5% DMSO (di-methyl-sulfoxide) and thawed in expansion medium. Fresh or frozen MSCs were used in experiments after 12 passages and no more than passage 20.

MSC Treatments for Assessing Neurogenic Potential In Vitro

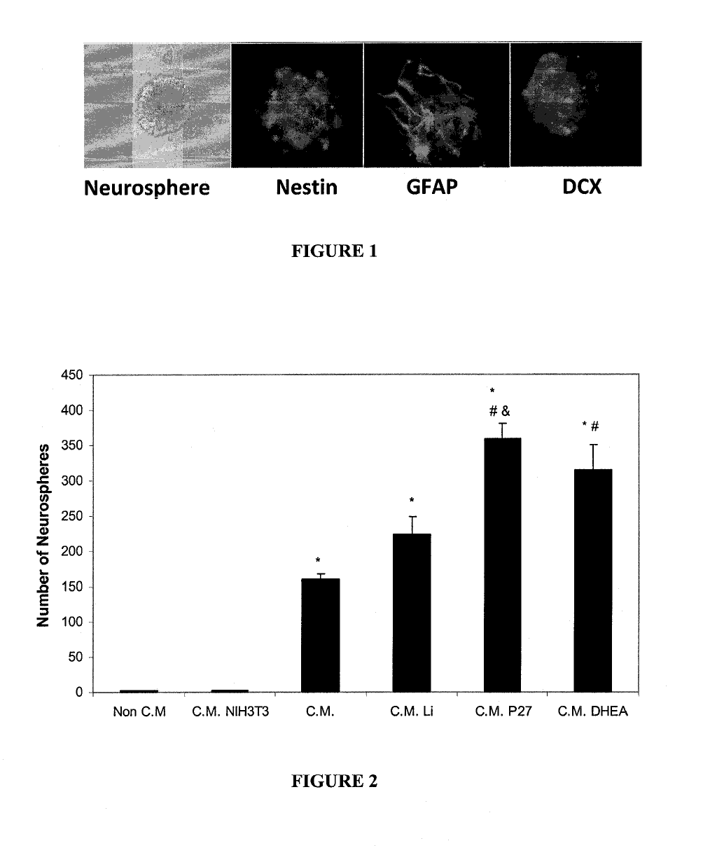

70-80% confluent cultures of bone marrow derived MSCs were cultured in vitro in Dulbecco's modified Eagle's medium (DMEM) without Fetal Bovine Serum (FBS) with and supplemented with the addition of one of the following compounds: None, LiCl (2 mM), PACAP27 (20 nM) or DHEA (1 mM). After 3 days medium was changed with fresh ingredients and cultures were allowed to grow for an additional 24 hours. After 24 hours plates were washed twice with PBS and conditioning medium (C.M.) containing none of the above compounds was placed for additional 24 hours. Conditioned medium harvested from these cultures after 24 hours was filtered in 0.2 um filter supplemented with 1% B27 nutrients mix and used as culture medium for a cell suspension of rat neonatal brain tissue in 24 well plates (10⁴ cells/well). A rat neonatal cortical cell suspension was obtained following incubation of dissected cortices obtained from sacrificed neonatal Sprague Dawley rats (Harlan, Jerusalem, Ill.) with 0.25% trypsin (Biological Industries) at 37° C. for 10 min. Neurotrophic factors present in the conditioned medium supported the growth of neurospheres in these cultures. Neurospheres are clones of differentiating neuroprogenitors present in the cell suspension. A typical neurosphere stained with neuronal and glial markers nestin; doublecortin (DCX); and glial fibrillary acidic protein (GFAP) is shown in FIG. 1.

The number of developing neurospheres in each conditioned medium type was counted after 5 days of culture and provided an indication of the neurogenic potential of the MSCs culture from which the CM was harvested.

PACAP Treatment of MSC Prior to In Vivo Injections

70-80% confluent cultures of bone marrow derived SABRA mice MSCs were cultured in expansion medium supplemented with PACAP27

(20 nM) for 3 days after which they were harvested for intravenouse injections (example 3).

SABRA mice MSCs were cultured in expansion medium supplemented with PACAP27 (20 nM) for 3 days after which fresh medium with fresh PACAP was added on day 4. Cultures were allowed to grow for additional 3 days and harvested on day 7 for ICV injections (Example 2).

ICR mice MSCs were cultured in expansion medium supplemented with PACAP27 (20 nM) for 3 days after which fresh medium with fresh PACAP was added on day 4. Cultures were allowed to grow for additional 24 hours and harvested on day 5 for ICV and IV injections (Example 6).

DiI Labeling of MSC Prior to In Vivo Transplantations

Suspended MSCs were labeled with DiI (1,1′-dioctadecyl-3,3,3′,3′-tetramethylindocarbocyanine perchlorate; Sigma) fluorescent dye in order to trace the migration of the cells in the brain. Briefly, trypsinized MSCs were suspended in PBS (10⁶ cells/ml) in the presence of DiI at a final concentration of 1 ug/ml and incubated for 10 min at 37° C. followed by 5 min at 4° C. and finally washed three times with PBS.

Amyloid Aggregates Preparation

Amyloid beta protein fragment 25-35 (Sigma) was dissolved in double distilled water at a final concentration of 1 mg/ml and was incubated in 37° C. for 4 days in order to form aggregates prior to ICV injections.

In Vivo Injections

Approximately 10⁵ DiI-labeled rat MSCs in a 10 μl volume were stereotactically injected into the right or both right and left lateral ventricle of anesthetized mice (coordinates referring to bregma A=−0.058 cm, L=±0.11 cm, V=−0.3 cm). Control animals were injected with vehicle only.

Intravenous injections were performed in non-anesthetized mice in the tail vein (200,000-500,000 cells/100 ul).

Prepulse Inhibition of the Acoustic Startle Response

Prepulse inhibition (PPI) test was conducted in startle device (Hamilton Kinder, Poway, Calif.) that consists of a plexiglas cylinder (10×10×5 cm) adapted for mouse size in order to restrict a mouse to stand up on its hind legs and enclosed in a ventilated sound attenuated compartment with a high-frequency loudspeaker producing all acoustic stimuli. The background noise was 65 dB. Movements within the cylinder were detected and transduced by a piezoelectric platform and recorded on a computer. Each trial was initiated with 5 minutes acclimation period followed by five startle pulse alone (ASR) successive testing trials and finishing with final five startle pulses alone. Auditory testing included startle pulse alone (ASR, 120 dB/40 msec) plus three different prepulse sessions in which either 20-msec-long 70, 74 and 82 dB stimuli were preceded the 120 dB pulse by 100 msec, with the alternate background noise measurement set as the baseline movements in the cylinder. All trials followed in a pseudo-random order and the average inter-trial interval (ITI) was 10 msec. PPI was defined as the percentage of the decreased in the ratio between the startle magnitude in the presence of the prepulse and the magnitude in the absence of the prepulse (100−(100× magnitude on prepulse+pulse trial/magnitude on pulse trial)).

Morris Water Maze

Morris water maze test measures the ability of rodents to learn, remember and recall spatial location of the hidden platform in a round water maze (Merenlender-Wagner, 2010 Morris 1984). The size of circular pool with opaque non-toxic black painted walls was of 100 cm diameter and 50 cm high. The apparatus was filled with warm water (24±1.0° C.). A non-visible escape platform (10 cm in diameter) constructed from black Perspex was placed approximately 0.5-1 cm below the water surface. Spatial cues were placed on the four walls of the pool and on this basis the platform was placed and moved during 5-6 experimental days. There were 3 cue trials for mouse to find the platform in one day. If within the trial (60 s) the platform was not found mouse were guided to the platform for 20 s and on the contrary if mouse found the platform before trial ended it was allowed to stay on the platform additional 20 s. After this time on the platform the mouse was removed from the pool, dried and returned to its home cage. Every day the platform was moved to the new place, close to the one of four points but not closer than 15 cm to the pool border. Latency time (i.e. the time that the mice spent in the water until reaching the platform) was manually recorded. For each day each mouse latency score was calculated as the mean result of its 3 trials during that day. A typical Morris maze test was conducted for 5 to 6 days.

Social Preference

The social interaction procedure was adapted from the social recognition test described by Engelmann et al (1995). Briefly the apparatus made as a large wooden open container (72×28×27 cm) enclosed were small wire cages (15×13×15 cm) on either ends of the box in which the juvenile mice were placed. The small wire cages were proposed to limit the mobility of juvenile mice while giving the possibility the adult test animal to have visual, olfactory and tactile access to the juveniles. For starting experiment adult tested mouse acclimates in the social interaction box for 20 min. After this period the learning session begins with the tested mouse allowed to explore a juvenile previously un-known mouse in one of the two small wire cages for 4 min. The adult tested animal was then returned to its home cage for 30 min. During the testing session or discrimination trial the adult tested mouse was returned to the experimental box where the now familiar and yet another novel mice were located in the wire cages. Within the trials the familiar juvenile was placed to the same small wire cage that the animal was initially located in during the learning session, while its place in the arena was switched. The adult tested mouse was allowed to explore both juveniles for additional 4 min. All sessions were videotaped and analyzed using Nodulus information technology Ethovision XT 7 software. The software was set to measure the interaction time with each of the juvenile mice (familiar and novel) by measuring the time spent by the tested mouse in the vicinity of each juvenile mouse cage. Recognition index was calculated as the ratio between the time spent for exploring the novel mouse with the total time spent by the tested mouse for exploring both familiar and novel mice in the test session.

Immunostaining

Immunoassays for the detection of the following proteins: nestin, doublecortin, GFAP, CD11b were performed on cell cultures and on frozen sections of mice brains. All primary antibodies were purchased from Abcam Inc. (Cambridge, Mass.) except CD11b (R&D systems) and secondary antibodies were purchased from KPL Inc. (Gaithersburg, Md.). Specifically, mice were anesthetized and perfused transcardially with 10 U/ml heparin followed by phosphate buffered saline (PBS, pH 7.4) followed by 4% paraformaldehyde (Sigma) in 0.1 M phosphate buffer (pH 7.4). Brains were removed, postfixed overnight and equilibrated in phosphate buffered 30% sucrose. Free-floating, 20-40 μm thick coronal hippocampal sections were prepared in a cryostat and stored in 0.1% sodium azide (Sigma) at 4° C. before immunofluorescence. Frozen tissue sections and cultured cells were washed with PBS, incubated in 0.1% Triton-X100 (Sigma) for 5 min and then blocked for 45 min with blocking solution (0.1% Triton-X100 and 5% BSA in PBS). Samples were then incubated with the following primary antibodies for 1 h at R.T.: mouse monoclonal anti-nestin (56 μg/ml), rabbit polyclonal anti-doublecortin (DCX) (1:5,000 dilution) and rabbit polyclonal anti-GFAP (1:100 dilution). Incubation with the appropriate secondary antibody (FITC goat anti-rabbit and FITC goat anti-mouse) at a 1:100 dilution was followed for 1 h at R.T. Between incubations, samples were washed 3 times with PBS. Sample assay results were visualized with a fluorescence microscope (TE2000-U, Nikon).

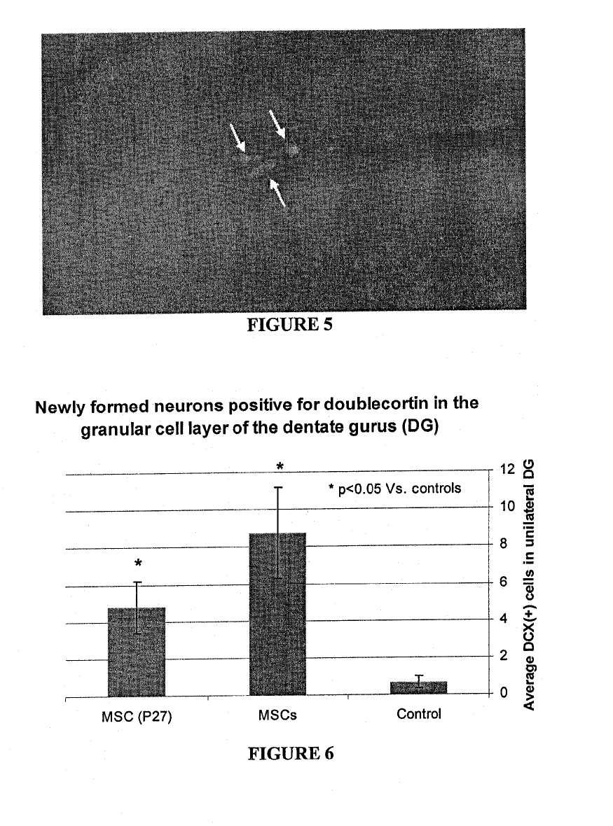

For immunohistochemistry (FIGS. 6, 7 and 13) an immunostaining kit (Zytochem plus HRP One-step polymer anti-Mouse/Rabbit/Rat) was used according to manufacturer protocol with the primary antibodies applied monoclonal anti mouse CD11b (1.25 μg/ml dilution) and rabbit polyclonal anti-doublecortin (DCX) (1:5,000 dilution) incubated overnight at 4° C.

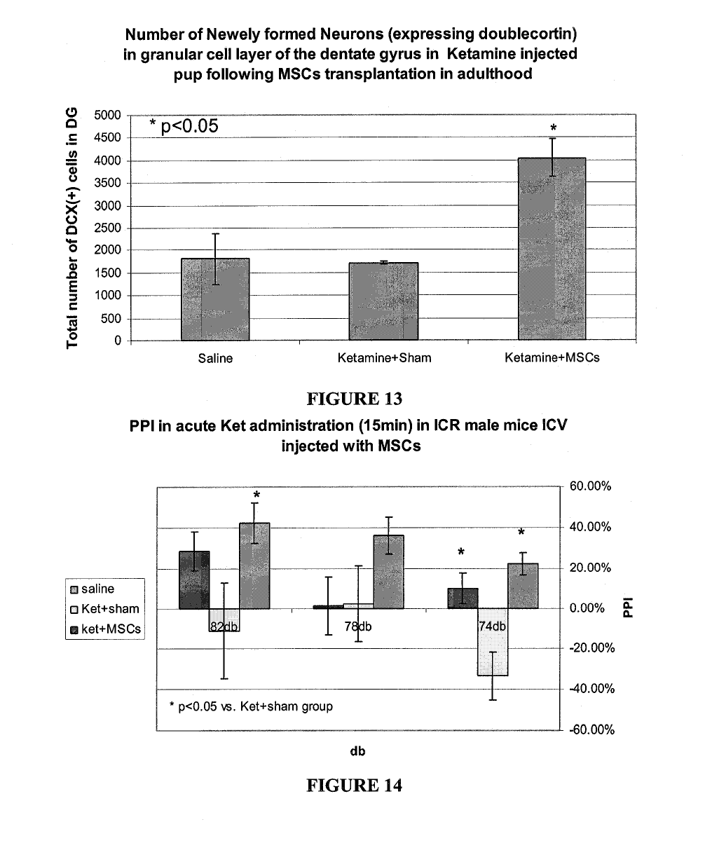

Quantification of the newly formed neurons in the dentate gyrus (DG) (FIGS. 6 and 13) was performed by counting doublecortin (DCX) positive cells in the granular cell layer on 6 representing sections of the dentate gyrus. For each mouse either the average cells/DG was calculated or total number of cells in DG was extrapolated from the average and the total length of the DG (total number of sections).

Statistics

Statistical tests applied for statistical significance were a two-tailed Student t-test and analysis of variance (ANOVA). Data are presented as mean±SE.

### Example 1

### Pre-Treatment of MSCs in Culture can Enhance Their Neurogenic Activity In Vitro

Bone marrow derived MSCs were cultured in vitro for 5-7 days in Dulbecco's modified Eagle's medium (DMEM) without Fetal Bovine Serum (FBS) and supplemented with the addition of one of the following compounds: None, LiCl (2 mM), PACAP27 (20 nM) or DHEA (1 mM). Conditioned medium (C.M.) harvested from these cultures after 24 hours was used as culture medium for a cell suspension of rat neonatal brain tissue (10⁴ cells/ml) supplemented with 1% B27 nutrients mix. Neurotrophic factors present in the conditioned medium supported the growth of neurospheres in these cultures. Neurospheres are clones of differentiating neuroprogenitors present in the cell suspension. A typical neurosphere stained with neuronal and glial markers nestin; doublecortin (DCX); and glial fibrillary acidic protein (GFAP) is shown in FIG. 1.

The number of developing neurospheres in each conditioned medium type was counted and provided an indication of the neurogenic potential of the MSCs culture from which the C.M. was harvested.

As shown in FIG. 2, a clear advantage was shown for MSC cultures treated with PACAP (P27) or DHEA over non-treated cultures (C.M. alone). NIH3T3 fibroblast C.M and non C.M. (i.e. basic culture medium) did not support the growth of neurospheres.

### Example 2

### Transplantation of Bone Marrow-Derived MSCs can Improve Cognitive Functions in Normal Mice

Female SABRA mice (10 w) were injected with 10⁵ cells/10 ul into the right lateral ventricle. A first group received MSCs; a second group was injected with MSCs previously cultured for one week with PACAP27 (P27) 20 nM. Control mice were injected with vehicle only. Two weeks after ICV injection, animals were tested for spatial learning (Morris water maze—MWM) as an indicator of cognitive function.

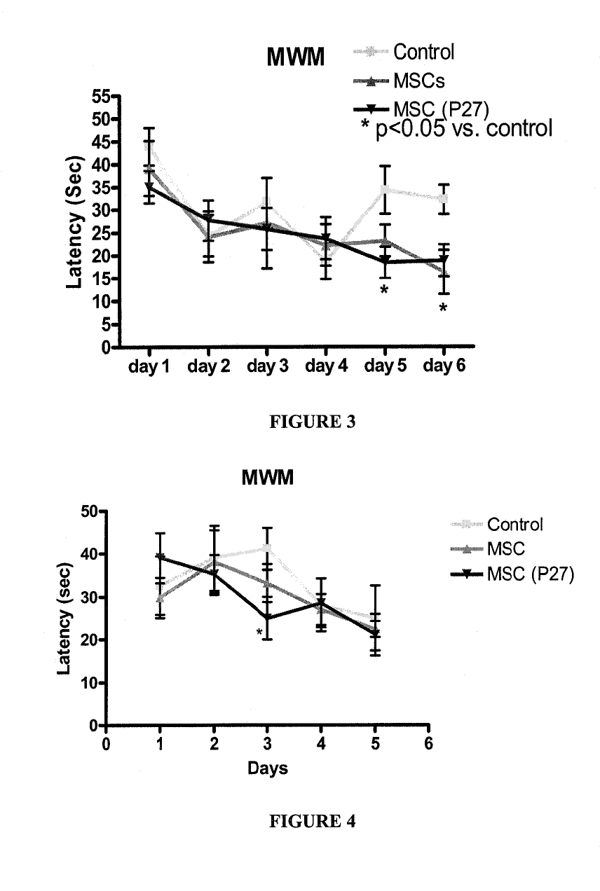

As shown in FIG. 3, a significant improvement was seen in the score obtained by mice injected with MSCs on day 6 as compared to controls. Mice injected with MSCs cultured with PACAP also obtained significantly higher scores than controls on days 5 and 6.

### Example 3

### PACAP Pre-Treated MSCs (“Activated” MSCs) Exhibit a Significant Effect on Cognitive Function when Administered Intravenously

Female SABRA mice (10 w) were injected with 500,000 cells/100 ul to the tail vein. A first group received MSCs; a second group was injected with MSCs previously cultured for 3 days with PACAP27 (P27) 20 nM. Control mice were injected with vehicle only. Two weeks after intravenous injection, animals were tested for spatial learning (Morris water maze—MWM) as an indicator of cognitive function.

As shown in FIG. 4, a significant improvement was seen in the score obtained by mice injected with MSCs cultured with PACAP as compared with controls on day 3.

As shown in FIG. 5, the presence of small numbers of engrafted PACAP-cultured stem cells was seen in the dentage gyrus. MSCs were labeled with dioctadecyl-tetramethylindocarbocyanine iodide (DiI) red fluorescence membrane stain in the sub granular zone of the dentate gyrus (indicated by arrows). Blue staining represents nuclei stained with 4′,6-diamidino-2-phenylindole (DAPI).

As shown in FIG. 6, mice implanted with MSCs (either with or without PACAP treatment) displayed increased neurogenesis as measured by the number of newly formed neurons in the granular cell layer of the dentate gyrus 3 weeks after transplantation. Newly formed neurons were detected following immunohistochemistry staining of frozen brain sections for doublecortin (DCX).

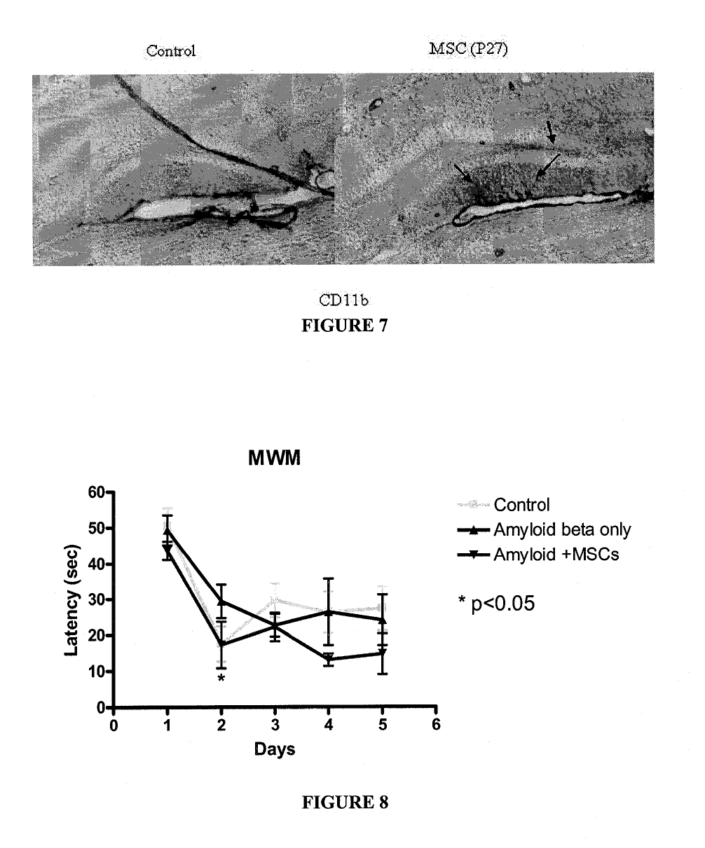

It is hypothesized that a possible explanation for the induced neurogenesis that was observed may be related to immunological changes in the brain in general and in the dentate gyrus in particular. As shown in FIG. 7, an increase was found in the presence of microglial cells, in the dentate gyrus of MSCs (P27) injected mice (detected with immunohistochemistry staining for CD11b, indicated by arrows). Thus, possible interaction of engrafted MSCs with the immune system locally in the brain or in other sites in the body, may also account for the observed changes in neurogenesis and behavior.

### Example 4

### Direct Injection of MSCs into the Lateral Ventricle in the Brain Results in Improved Cognitive Function in a Murine Model of Acute Alzheimer's Disease

Male ICR mice (6 w) were injected bilaterally in the lateral ventricle with amyloid beta aggregates prepared from amyloid beta peptide (25-35). Amyloid beta aggregates (3 ug) were ICV injected followed five minutes later by an injection with 10⁵ MSCs or vehicle in each lateral ventricle. Control animals were injected with vehicle (no amyloid and no MSCs). Animals were tested for spatial learning (Morris water maze—MWM) as an indicator of cognitive function.

As shown in FIG. 8, a significant improvement in the scores obtained by Control and Amyloid+MSCs groups was found as compared to mice injected with amyloid only on day 2.

### Example 5

### Direct Injection of MSCs into the Lateral Ventricle Improved Spatial Learning, Social Preference and Pre-Pulse Inhibition in Murine Models of Schizophrenia

The present inventors used two models for schizophrenia in mice, both induced by the NMDA antagonist ketamine. Two distinct features impaired in animal models of schizophrenia are pre-pulse inhibition plus acoustic startle response, and social interaction (impaired preference of interaction with novel mice over known mice, reflecting cognitive and affective behaviors).

Model I:

A developmental model which represents a defect resulting from ketamine administration during development and its effect in adulthood. In this model, neonatal female ICR pups were injected subcutaneously with ketamine 50 mg/kg at days 6, 7 and 8 days postnatal. At adulthood (22 weeks of age) mice were injected with MSCs (10⁵ cells/10 μl) into the right lateral ventricle. Animals were tested for social preference (social discrimination assay, novelty preference) 4 weeks before injection of MSCs and two weeks after injection.

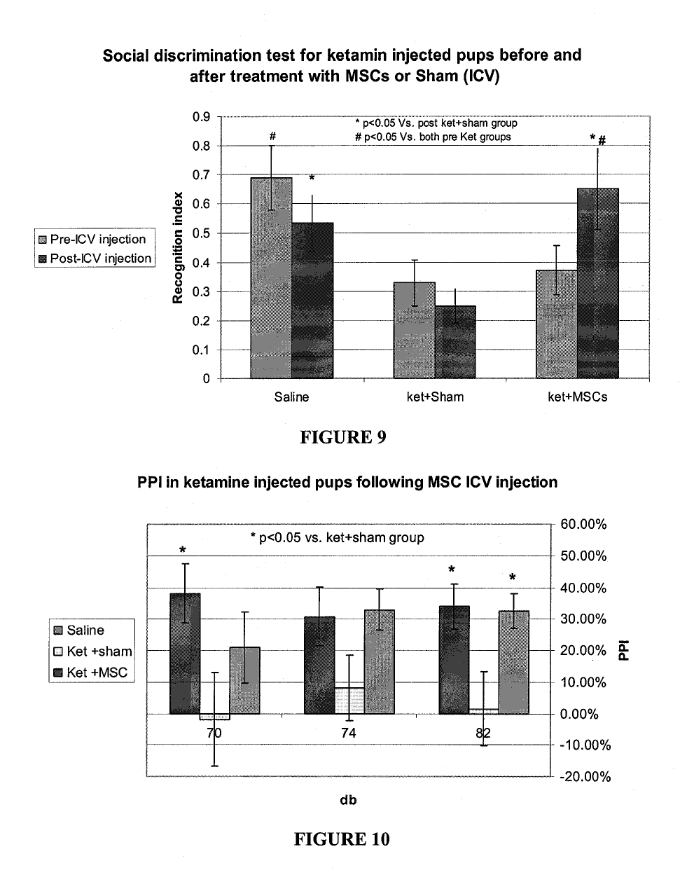

As shown in FIG. 9, an impaired preference for novel mice (low recognition index) was seen in the ketamine-injected groups before ICV injections. This impairment was corrected following ICV injection of MSCs but not in sham operated mice.

Pre-pulse inhibition assay was carried out 3 weeks after transplantation. As shown in FIG. 10, a significant increase in pre-pulse inhibition (PPI) was seen in ketamine-exposed mice later injected with MSCs as compared to sham mice at 70 db pre-pulse and 82 db pre-pulse. PPI scores for MSCs treated mice resembled those of normal mice that were injected as pups with saline and not ketamine.

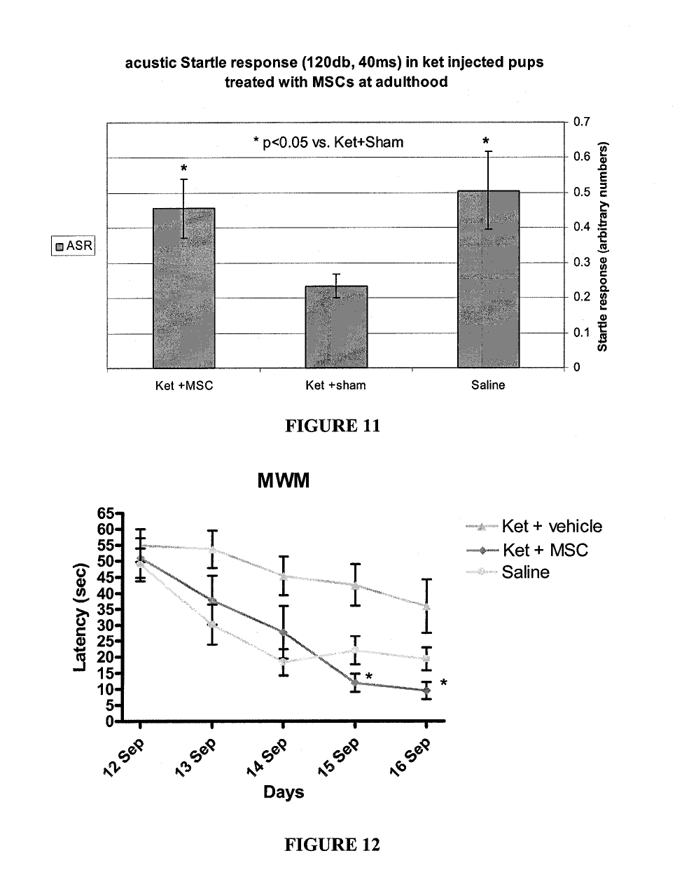

As shown in FIG. 11, the acoustic startle response (ASR) to high volume pulse stimulus (120 db, 40 msec) with no pre-pulse is also impaired in ketamine injected mice (ket+sham) compared with mice not exposed to ketamine (saline). ASR was restored to normal scores in MSCs treated mice (ket+MSCs).

In a further experiment, it was shown (FIG. 12) that pups injected with ketamine (50 mg/kg) at day 10 only and which exhibited low PPI scores at age 7 w, also had impaired cognitive functions i.e. decreased spatial learning ability in the Morris water maze (age 10 w). These mice received MSCs ICV injections (10⁵ cells/10 ul) or sham injections (ket+vehicle). Two weeks post ICV injection, MSCs injected mice showed improved scores as control (saline and not ketamine injected mice), scoring better than ketamine injected mice that underwent sham ICV injections.

Post-mortem analysis, as represented in the graph of FIG. 13, showed increased neurogenesis in ketamine+MSCs group compared with the sham operated mice and control (saline) mice as reflected by the increased number of new neurons (i.e. DCX positive cells in immunohistochemistry) in the granular cell layer of the dentate gyrus. These results signify the importance of hippocampal neurogenesis in mediating the behavioral change observed in these mice.

Model II:

An acute exposure of adult ICR male mice (13 weeks old) to ketamine (20 mg/kg) and its effect 15 minutes thereafter on pre-pulse inhibition. Two weeks before ketamine exposure, mice were injected with MSCs (10⁵ cells/10 ul) or vehicle into the right lateral ventricle. As controls, age-matched mice injected with saline and not ketamine were applied. As shown in FIG. 14, impaired PPI was seen in ketamine-exposed mice (ket+sham) compared with normal mice injected with saline on 72 db and 82 db pre-pulses. MSCs ICV injection two weeks before the assay significantly improved PPI in the 72 db pre-pulse and provided values closer to normal mice (saline).

### Example 6

### MSCs Administration, Following PACAP Treatment in Culture, Exhibit Long Term Improvement in Social Preference and Pre-Pulse Inhibition in Murine Model of Schizophrenia

MSC treatment with PACAP was accomplished by culturing bone marrow MSC at 70-80% confluence with complete culture media supplemented with 20 nM PACAP27 for 3 days. Medium was then replaced with new medium containing fresh PACAP27. Cells were allowed to culture for additional day before harvesting for transplantations.

Neonatal female ICR pups were injected subcutaneously with ketamine 50 mg/kg at days 6, 7 and 8 days postnatal (as in Model I above). At adulthood (6 months of age) mice were sub-grouped and treated accordingly:
Group 1: Saline (non ket)—control animals not subjected to ketamine postnatally.
Group 2: Ket control—postnatal ketamine injected animals not treated at all (injected with vehicle at adulthood).
Group 3: Ket+MSC(icv)—postnatal Ketamine injected animals treated with MSC injected (10⁵ cells/10 microlitter) into the right lateral ventricle.
Group 4: Ket+MSCp(icv)—postnatal ketamine injected animals treated with PACAP treated MSC injected (10⁵ cells/10 microlitter) into the right lateral ventricle.
Group 5: Ket+MSC(iv)—postnatal Ketamine injected animals injected intravenously with MSC (200,000 cells/100 microlitter).
Group 6: Ket+MSCp(iv)—postnatal Ketamine injected animals injected intravenously with PACAP treated MSC (200,000 cells/100 microlitter).
Group 7: Clozapine—postnatal Ketamine injected animals injected intraperitoneally with anti-psychotic drug clozapine for 12 days. At day one 10 mg/kg, at day two 5 mg/kg and for the following ten days 3 mg/kg.

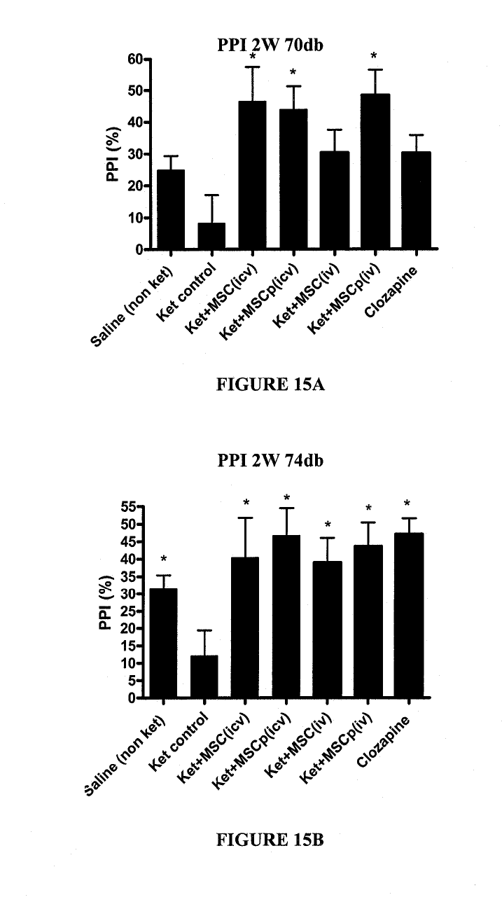

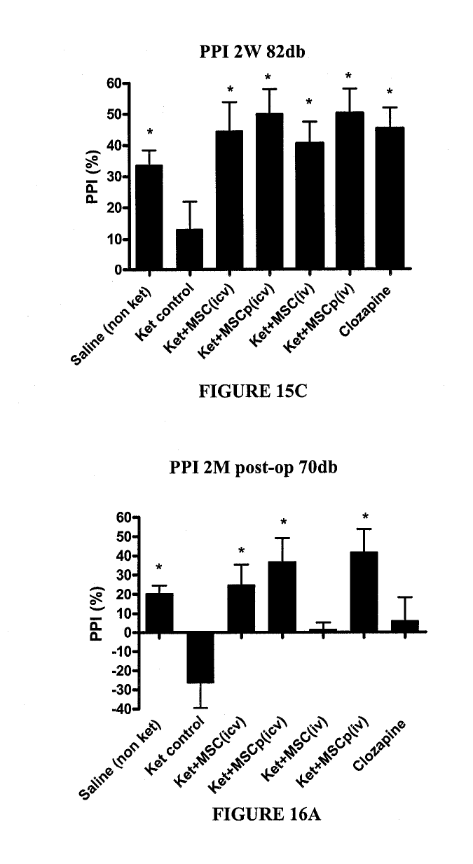

FIG. 15 shows the PPI measurements obtained for all groups 2 weeks (2 W) after injections and for the clozapine at day 10 of the treatment course. FIGS. 15 A, B and C represent prepulse at the intensity of 70 db, 74 db and 82 db respectively. Note, the significant increase in PPI in all treatment groups essentially.

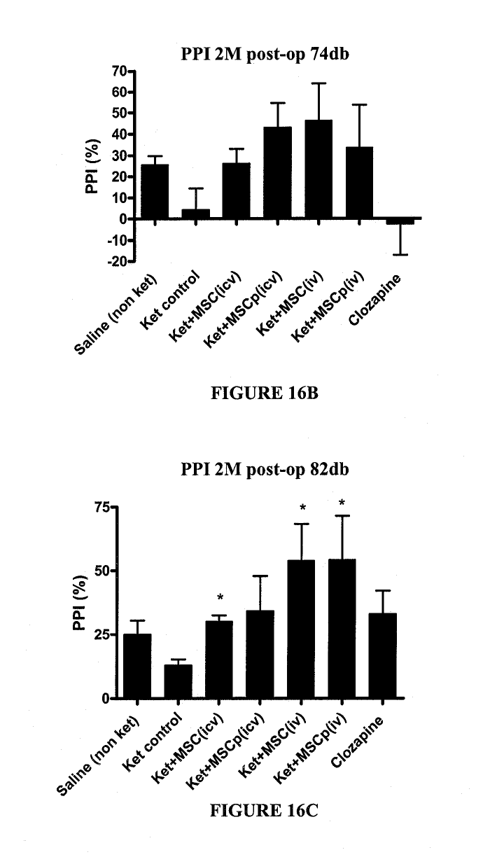

FIG. 16 shows the PPI measurements obtained for all groups 2 months after injections (2M post-op). FIGS. 16 A, B and C represent prepulse at the intensity of 70 db, 74 db and 82 db respectively. Note, the significant increase in PPI in particularly in the ket+MSC(icv) and ket+MSCp(iv) groups and to a lesser extent ket+MSCp(icv) and ket+MSC(iv). Previous Clozapine treatment at that time (6 weeks after the course ended) had no significant effect.

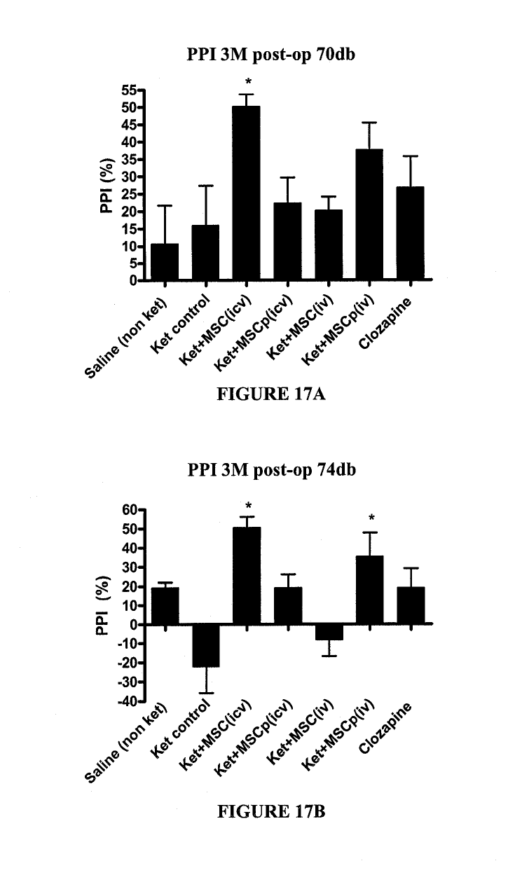

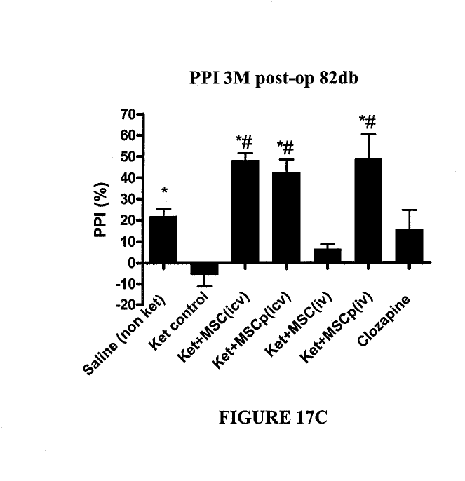

FIG. 17 shows the PPI measurements obtained for all groups 3 months after injections (3M post-op). FIGS. 17 A, B and C represent prepulse at the intensity of 70 db, 74 db and 82 db respectively. Note, the significant increase in PPI in particularly in the ket+MSC(icv) and ket+MSCp(iv) groups and to a lesser extent ket+MSCp(icv). Previous Clozapine treatment at that time (2.5 months after the treatment course ended) had no significant effect.

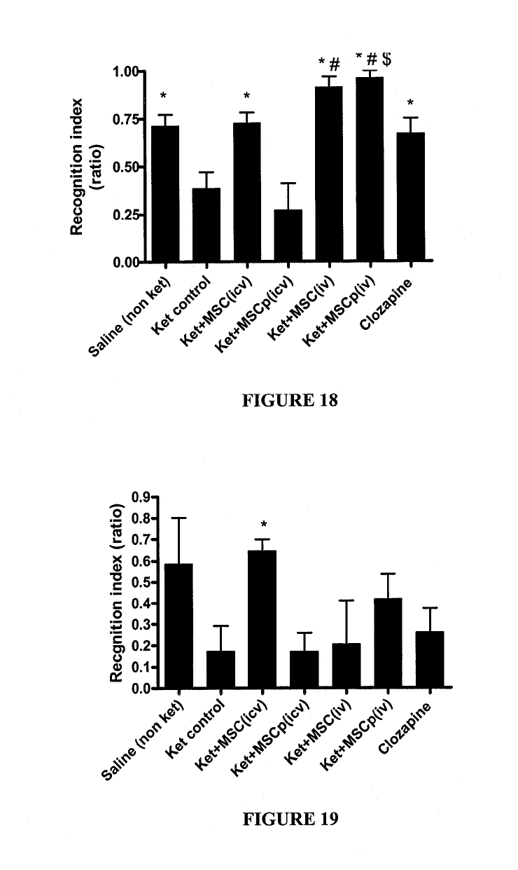

Social interaction assay measuring social preference was tested 2 weeks and 3 months after MSC injections. Two weeks post treatment all treatment groups including clozapine group (day 7 of treatment course), but except ket+MSCp(icv) group, demonstrated improved preference for the novel mouse (increased recognition index) compared with Ket control group. Moreover, ket+MSCp(iv) obtained the best results significantly surpassing clozapine and MSC(icv) treatmens (FIG. 18).

At 3 months after treatment only ket+MSC(icv) maintained its improved social preference (FIG. 19).

The results shown in FIGS. 15-19 show that MSCs injected directly into the lateral ventricle or intravenously, following PACAP treatment in culture, exhibit long term improvement in social preference and pre-pulse inhibition in murine model of schizophrenia surpassing the effect of a 12 days course treatment with clozapine.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

What is claimed is:

1. A method of improving cognitive function of a subject, the method comprising pretreatment of mesenchymal stem cells (MSCs) with at least one agent selected from the group consisting of pituitary adenylate cyclase-activating polypeptide (PACAP) or analogs or fragments thereof, dehydroepiandrosterone (DHEA) and lithium chloride (LiCl); and administering to said subject a pharmaceutically effective amount of the MSCs thereby improving the cognitive function of said subject.

2. The method of claim 1, wherein the at least one agent is PACAP, said PACAP comprising an amino acid sequence selected from the group consisting of SEQ ID NOs: 1, 2, 3, 4, 5, 6, 7, 8, 10 and 11.

3. The method of claim 1, wherein said PACAP has an amino acid sequence selected from the group consisting of SEQ ID NOs: 1, 2, 3, 4, 5, 6, 7, 8, 10 and 11.

4. The method of claim 1, wherein said PACAP has the amino acid sequence selected from SEQ ID NO: 1, 2, 3 and 4.

5. The method of claim 1, wherein said PACAP is PACAP-27 having an amino acid sequence as set forth in SEQ ID NO:1.

6. The method of claim 1, wherein the MSCs are bone-marrow derived MSCs or wherein the MSCs are human cells.

7. The method of claim 1, wherein said subject is suffering from a neurodegenerative or neuropsychiatric disease.

8. The method of claim 1, wherein the neurodegenerative disease is Alzheimer's disease.

9. The method of claim 1, wherein the neuropsychiatric disease schizophrenia.

10. The method of claim 1, wherein said method has a long term effect in improving the cognitive function of said subject.

11. The method of claim 1, wherein the long term effect is for at least 2 months following administration.

12. The method of claim 1, wherein said MSCs administration is intracerebroventricular administration or intravenous administration.

13. A method for generating mesenchymal stem cells (MSCs) comprising culturing MSCs in the presence of an effective amount of at least one agent selected from the group consisting of pituitary adenylate cyclase-activating polypeptide (PACAP) or analogs or fragments thereof, dehydroepiandrosterone (DHEA) and lithium chloride (LiCl).

14. The method of claim 13, thereby enhancing the neurogenic activity of said MSCs.

15. The method of claim 13, wherein said MSCs are useful for treating a neurodegenerative or neuropsychiatric disease or for improving cognitive function of a subject.

16. The method of claim 13, wherein said MSCs are bone-marrow derived MSCs.

17. The method of claim 13, wherein the at least one agent is PACAP, said PACAP comprising an amino acid sequence selected from the group consisting of SEQ ID NOs: 1, 2, 3, 4, 5, 6, 7, 8, 10 and 11.

18. The method of claim 13, wherein said PACAP has the amino acid sequence of SEQ ID NO: 1.

19. A method of treating schizophrenia in a subject in need thereof, the method comprising administering to said subject a pharmaceutically effective amount of MSCs.

20. The method of claim 19, comprising pretreatment of the MSCs with an effective amount of PACAP or analogs or fragments thereof.


SEQUENCE LISTING (11 sequences)

SEQ ID NO: 1 - protein, 27 aa, Artificial
  Synthetic peptide
HSDGIFTDSYSRYRKQMAVKKYLAAVL

SEQ ID NO: 2 - protein, 38 aa, Artificial
  Synthetic peptide
HSDGIFTDSYSRYRKQMAVKKYLAAVLGKRYKQRVKNK

SEQ ID NO: 3 - protein, 22 aa, Artificial
  Synthetic peptide
FTDSYSRYRKQMAVKKYLAAVL

SEQ ID NO: 4 - protein, 33 aa, Artificial
  Synthetic peptide
FTDSYSRYRKQMAVKKYLAAVLGKRYKQRVKNK

SEQ ID NO: 5 - protein, 28 aa, Artificial
  Synthetic peptide
HSDAVFTDNYTRLRKQMAVKKYLNSILN

SEQ ID NO: 6 - protein, 30 aa, Artificial
  Synthetic peptide
HSDAVFTENYTKLRKQLAAKKYLDLKKGGT

SEQ ID NO: 7 - protein, 27 aa, Artificial
  Synthetic peptide
HSDAVFTENYTKLRKQLAAKKYLDLKK

SEQ ID NO: 8 - protein, 27 aa, Artificial
  Synthetic
HSDAVFTNSYRKVLKRLSARKLLQDIL

SEQ ID NO: 9 - protein, 176 aa, Homo sapiens
MTMCSGARLALLVYGIIMHSSVYSSPAAAGLRFPGIRPEEEAYGEDGNPLPDFDGSEPPGAGSPASAPRAAAAWYRPAGRRDVAHGILNEAYRKVLDQLSAGKHLQSLVARGVGGSLGGGAGDDAEPLSKRHSDGIFTDSYSRYRKQMAVKKYLAAVLGKRYKQRVKNKGRRIAYL

SEQ ID NO: 10 - protein, 30 aa, Artificial; identical to SEQ ID NO: 6
  Synthetic peptide

SEQ ID NO: 11 - protein, 27 aa, Artificial
  Synthetic peptide
HSDAVFTENYTKLRKQLAAKKYLDLKK